(19) United States
(12) Patent Application Publication
(10) Pub. No.: US 2026/0062814 A1
(43) Pub. Date: Mar. 5, 2026

(54) NOVEL AND DIRECT SYNTHESIS METHOD FOR PYRIDINIUM SALTS BASED ON ALKYL EPOXIDES AND THEIR USE

(71) Applicant: Saudi Arabian Oil Company, Dhahran (SA)

(72) Inventors: Muhammad Imran Ulhaq, Dhahran (SA); Qasim Saleem, Khobar (SA)

(73) Assignee: Saudi Arabian Oil Company, Dhahran (SA)

(21) Appl. No.: 18/824,556

(22) Filed: Sep. 4, 2024

(51) Int. Cl. C23F 11/14; C07D 213/04

(52) U.S. Cl. CPC C23F 11/149; C07D 213/04

(57) ABSTRACT

A formulation for inhibiting metal corrosion includes a solution comprising a pyridine salt compound comprising a Formula (I), as described herein, dissolved in a non-aqueous solvent. A method of making a pyridinium salt-based solution comprises reacting a pyridine having Formula (II), described herein, or a salt thereof with an epoxide having a Formula (III), described herein, in the presence of an acid and a non-aqueous solvent.

### TECHNICAL FIELD

Embodiments of the present disclosure generally relate to formulations and processes for inhibiting corrosion and, more specifically, to corrosion inhibitors having compounds comprising a pyridinium salt compound dissolved in a non-aqueous solvent and their use for mitigating corrosion in corrosive environments.

### BACKGROUND

Corrosion is the irreversible interfacial reaction of a material with its environment, which results in the consumption of the material. According to a recent report by the National Association of Corrosion Engineers (NACE), the annual worldwide cost of corrosion is over 2.5 trillion U.S. dollars. In the oil and gas industry, corrosion can inflict severe damage on the internal walls of production and transportation pipelines, which are mostly steel based materials. Specifically, corrosion can lead to pipeline leakages and, in some cases, bursting. Such damage can result in high maintenance costs, discontinuity of operations, and low capacity production.

Sources of corrosion of metal surfaces include dissolved gasses, such as carbon dioxide (CO₂), which causes “sweet corrosion,” and hydrogen sulfide (H₂S), which causes “sour corrosion.” Once dissolved in water, both CO₂ and H₂S behave like weak acids and can provide oxidizing power, which promotes steel corrosion. The more dominant of either sweet or sour corrosion in oilfield pipelines depends on the relative abundance of each gas in the environment. To mitigate sweet and sour corrosion during oil and gas production, transportation, and processing, corrosion inhibitors are commonly injected into pipeline fluids.

Mitigating pipeline corrosion in wet sour environments is a particular challenge for the oil and gas industry. Some of the most common corrosion inhibitor formulations are film formers based on nitrogen-containing compounds. They retard metallic corrosion by adsorbing onto the metal surface to create inhibitor barriers between the metal surface and the corrosive environment. Notable classes of corrosion inhibitor formulations include nitrogen-based compounds such as imidazolines, amines, and quaternary ammonium salts. Commonly used corrosion inhibitor formulations to mitigate corrosion in a wet sour environment include alkyl pyridinium benzyl quaternary ammnonium salts (APBQA). APBQAs form a relatively weak film barrier between the fluid phase and the metal surface. Thus, a high dosage of APBQA based corrosion inhibitor formulations is needed to significantly mitigate corrosion in a wet sour environment.

There is a need for improved corrosion inhibitor formulations.

### SUMMARY

Embodiments of the corrosion inhibitor formulations described herein meet this need through the inclusion of compounds comprising a pyridinium salt compound dissolved in a non-aqueous solvent. According to one or more embodiments of the present disclosure, a formulation for inhibiting metal corrosion includes a solution comprising a pyridine salt compound of Formula (I)

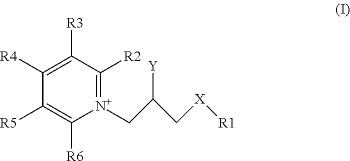

wherein R₁-R₆ independently comprise hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide; and X and Y independently comprise a heteroatom selected from oxygen, nitrogen, or sulfur, or a heterocarbyl comprising one or more heteroatoms selected from oxygen, nitrogen, or sulfur.

According to one or more embodiments of the present disclosure, a method of making a pyridinium salt-based solution comprises reacting a pyridine comprising Formula (II) or a salt thereof

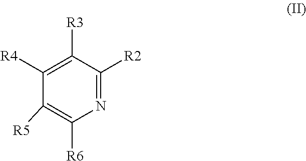

with an epoxide comprising Formula (III)

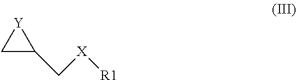

in the presence of an acid and a non-aqueous solvent to produce the pyridinium salt-based solution, comprising a pyridine salt compound of Formula (I) dissolved in the non-aqueous solvent, wherein R₂, R₃, R₄, R₅, and R₆ are independently hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide; R₁ is a substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, haloalkyl, azidoalkyl, amide, amino acid, or peptide; X and Y independently comprise a heteroatom selected from oxygen, nitrogen, or sulfur, or a heterocarbyl comprising one or more heteroatoms selected from oxygen, nitrogen, or sulfur.

Additional features and advantages of the described embodiments will be set forth in the detailed description which follows, and in part will be readily apparent to those skilled in the art from that description or recognized by practicing the described embodiments, including the detailed description which follows, the claims, as well as the appended drawings.

It is to be understood that both the foregoing general description and the following detailed description describe various embodiments and are intended to provide an overview or framework for understanding the nature and character of the claimed subject matter.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 graphically depicts the ¹HNMR spectrum of a synthesized 1-(2-hydroxyalkyl) pyridinium compound (1-(2-hydroxydodecyl)pyridinium chloride) prepared in water at (A) 23 hours, (B) 7 hours, and (C) 4 hours, according to one or more embodiments shown and described herein;

FIG. 2 graphically depicts the ¹H NMR spectrum of a synthesized 1-(2-hydroxyalkyl) pyridinium compound (1-(2-hydroxydodecyl)pyridinium chloride) prepared in monoethylene glycol at (A) 23 hours, (B) 7 hours, and (C) 4 hours, according to one or more embodiments shown and described herein;

FIG. 3 is a mass analysis ESI spectrum of an embodiment of an active component comprising 1-(2-hydroxy dodecyl) pyridinium salt according to embodiments described herein;

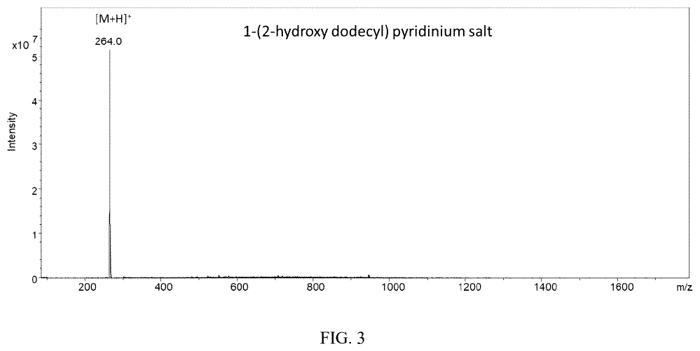

FIG. 4 is a mass analysis ESI spectrum of an embodiment of an active component comprising 1-[3-(Dodecyloxy/tetradecyloxy)-2-hydroxypropyl] pyridinium according to embodiments described herein; and

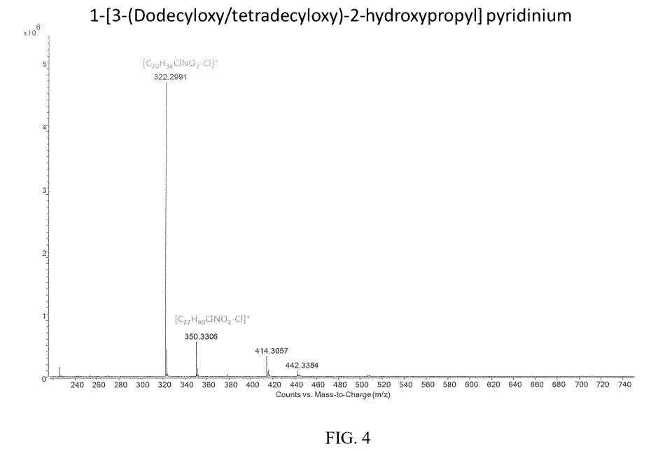

FIG. 5 is a flow chart comparing the present embodiment of producing a corrosion inhibitor formulation to the conventional process for achieving a corrosion inhibitor formulation.

Reference will now be made in greater detail to various embodiments, some embodiments of which are illustrated in the accompanying drawings.

### DETAILED DESCRIPTION

Embodiments of the present disclosure are directed to formulations and methods for inhibiting metal corrosion.

As used herein, the term “active component” and “active compound” is defined as a component in a corrosion inhibitor formulation that acts to mitigate corrosion of a material. Active components are distinguished from inactive components, which mainly serve as a vehicle to convey an active component. Corrosion inhibitor formulations according to embodiments described herein may include one or more active components.

As used herein, “the active component” refers to the component in a corrosion inhibitor formulation that acts to mitigate corrosion of a material to a greater extent than any other individual component in the corrosion inhibitor formulation.

As used herein, “parts per million” or “ppm” refers to parts per million by weight.

It was discovered that embodiments of formulations described herein that include a pyridinium salt compound dissolved in a non-aqueous solvent exhibit superior corrosion inhibiting efficiency compared with conventional corrosion inhibitors. Without being bound by theory, it is believed that corrosion inhibitors including a pyridinium salt comprising Formula (I) dissolved in a non-aqueous solvent may be used directly at the field without the need for any further treatment as corrosion inhibitor, or, may be part of a formulation without any purification or separation. According to embodiments described herein, pyridine-epoxide salts prepared in non-aqueous solutions may be used directly as effective corrosion inhibitors without the need for pyridinium-epoxide salt recovery, extraction, or separation. Specifically, it is believed that non-aqueous solvents including diethylene glycol monoethyl ether, 2-butoxyethanol, and preferably monoethylene glycol may facilitate the reaction kinetics of the synthesis of pyridine-epoxide salts just as efficiently as aqueous media because the non-aqueous solvents maintain the reaction mixture and final product in solution phase during the conversion process. Compared with that prepared in aqueous reaction media such as water, the formulations as described herein exhibit similar performance with respect to chemical and physical properties including structure, inhibition efficiency, and corrosion rate. Furthermore, conventional corrosion inhibitors prepared in aqueous reaction media have not been enabled to control the pyridinium-epoxide salt ratio at the final corrosion inhibitor formulation as accomplished by embodiments described herein. As such, it is believed that corrosion inhibitor formulations as described herein are comparable with conventional corrosion inhibitors at mitigating corrosion of metal surfaces.

According to embodiments, a formulation for inhibiting metal corrosion may include a solution comprising a pyridine salt compound of Formula (I) dissolved in a non-aqueous solvent.

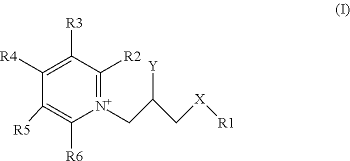

According to embodiments, R₁-R₆ in Formula (I) independently comprise hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide. In embodiments, R₂, R₃, R₄, R₅, and R₆ in Formula (I) are independently hydrogen and R₁ in Formula (I) is an unsubstituted C₅ to C₁₅ alkyl. In specific embodiments, R₂, R₃, R₄, R₅, and R₆ in Formula (I) are independently hydrogen and R₁ in Formula (I) is an unsubstituted C₉ alkyl or an unsubstituted C₁₄ alkyl.

According to embodiments, X and Y in Formula (I) independently comprise a heteroatom selected from oxygen, nitrogen, or sulfur, a heterocarbyl comprising one or more heteroatoms selected from oxygen, nitrogen, or sulfur. In specific embodiments, Y in Formula (I) is a hydroxyl group.

According to embodiments, the pyridine salt compound having Formula (I) may comprise at least one halogen selected from a group comprising chlorine or bromine. In specific embodiments, the pyridine salt compound having Formula (I) comprises chlorine.

In embodiments, the compound comprising Formula (I) is soluble in water.

In embodiments, the compound comprising Formula (I) has an average molecular mass ranging from 100 m/z to 1500 m/z. According to one or more embodiments, the compound is present in the formulation at an average molecular mass of from 100 m/z to 1300 m/z, from 100 m/z to 1100 m/z, from 100 m/z to 900 m/z, from 100 m/z to 700 m/z, from 100 m/z to 500 m/z, from 100 m/z to 300 m/z, from 200 m/z to 1400 m/z, from 200 m/z to 1200 m/z, from 200 m/z to 1000 m/z, from 200 m/z to 800 m/z, from 200 m/z to 600 m/z, or even from 200 m/z to 400 m/z.

In some embodiments, the non-aqueous solvent may comprise at least one alcohol. In further embodiments, the alcohol may comprise monoethylene glycol (MEG), diethylene glycol monoethyl ether (DGME), 2-butoxyethanol, or a combination thereof. In specific embodiments, the non-aqueous solvent is monoethylene glycol.

In embodiments, the compound comprising Formula (I) may be dissolved in a non-aqueous solvent at a temperature ranging from 40° C. to 80° C. and left under mixing for up to 6 hours. In embodiments, the compound comprising Formula (I) may be dissolved in a non-aqueous solvent at a temperature of from 40° C. to 70° C., from 40° C. to 60° C., from 40° C. to 50° C., from 50° C. to 80° C., from 50° C. to 70° C., from 50° C. to 60° C., from 60° C. to 80° C., or even from 60° C. to 70° C.

In some embodiments, a method of making a pyridinium salt-based solution comprises reacting a pyridine comprising Formula (II) or a salt thereof, with an epoxide comprising Formula (III) in the presence of an acid and a non-aqueous solvent.

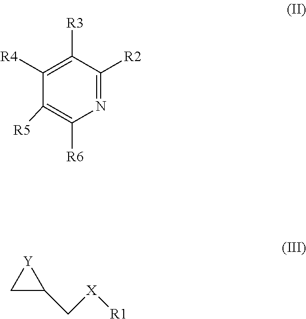

In embodiments, R₂, R₃, R₄, R₅, and R₆ in Formula (II) are independently hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide.

According to one or more embodiments, R₁ is a substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, haloalkyl, azidoalkyl, amide, amino acid, or peptide. In some embodiments, X and Y independently comprise a heteroatom selected from oxygen, nitrogen, or sulfur, or a heterocarbyl comprising one or more heteroatoms selected from oxygen, nitrogen, or sulfur.

In some embodiments, R₂, R₃, R₄, R₅, and R₆ in Formula (II) are independently hydrogen and R₁ of Formula (I) is an unsubstituted C₅ to C₁₅ alkyl.

In some embodiments, the acid comprises hydrochloric acid, sulfuric acid, phosphoric acid, hydrobromic acid, acetic acid, nitric acid, or a combination thereof. In embodiments, the acid comprises hydrochloric acid, phosphoric acid, sulfuric acid, or a combination thereof. In specific embodiments, the acid comprises hydrochloric acid.

In embodiments, the formulation may be used to mitigate corrosion of a material. In some embodiments, the formulations may be used to mitigate corrosion of a metallic surface. In other embodiments, the formulations may be used to mitigate corrosion of a metallic surface, such as a metallic surface under wet sour crude conditions. According to one or more embodiments, the formulations may be included in a sour well fluid to mitigate the corrosion of surfaces exposed to the sour well fluid. In embodiments, the sour well fluid may further comprise petroleum hydrocarbons.

In embodiments, a process for mitigating corrosion of a metallic surface may include contacting the metallic surface with a solution comprising the compound at a concentration by weight of 0.1 parts per million to 100 parts per million, 0.2 parts per million to 50 parts per million, 0.2 parts per million to 40 parts per million, 0.2 parts per million to 30 parts per million, 0.2 parts per million to 20 parts per million, 0.2 parts per million to 10 parts per million, 0.3 parts per million to 8 parts per million, 0.4 parts per million to 6 parts per million, 0.5 parts per million to 5 parts per million, 0.5 parts per million to 2 parts per million, or 1 part per million to 2 parts per million of the well fluid. According to embodiments, the metallic surface comprises steel. In some embodiments, the metallic surface comprises carbon steel. In embodiments, the metallic surface may be contacted with the corrosion inhibitor at least one time, at least two times, at least three times, at least four times, or even at least five times. It should be understood that when the metallic surface is contacted with the corrosion inhibitor multiple times, each contacting may be accomplished using the same or different concentrations of the compound described herein.

In some embodiments, the compound described herein may be used as an active component in a formulation for inhibiting corrosion. According to one or more embodiments, a corrosion inhibitor formulation comprises the compound and water. In some embodiments, the corrosion inhibitor formulation further comprises a synergist, a nonionic surfactant, imidazoline, a supporting component, a secondary solvent, a coupling agent, an ethoxylated amine, or a combination thereof.

According to one or more embodiments, the compound is present in the corrosion inhibitor formulation at a concentration of from 0.5 to 60 weight percent, 0.5 to 59 weight percent, 0.6 to 50 weight percent, 0.7 to 50 weight percent, 0.8 to 50 weight percent, 0.9 to 50 weight percent, 1.0 to 50 weight percent, 1 to 40 weight percent, 2 to 40 weight percent, 3 to 40 weight percent, 4 to 40 weight percent, 5 to 40 weight percent, 5 to 35 weight percent, 5 to 30 weight percent, 5 to 25 weight percent, 5 to 20 weight percent, or 10 to 20 weight percent. In embodiments, water is present in the corrosion inhibitor formulation at a concentration of from 0 to 90 weight percent, 1 to 89 weight percent, 5 to 85 weight percent, 10 to 80 weight percent, 20 to 70 weight percent, 30 to 60 weight percent, 40 to 50 weight percent, 40 to 90 weight percent, 50 to 90 weight percent, 50 to 80 weight percent, 60 to 80 weight percent, or 65 to 75 weight percent.

In some embodiments, the corrosion inhibitor formulation includes a synergist. The synergist may act to facilitate the adsorption of an active component comprising a quaternary ammonium or pyridinium substituent onto the surface of a metal. Without being bound by theory, it is believed that the synergist adsorbs onto a metal surface while also attracting an active component comprising a quaternary ammonium or pyridinium substituent. In some embodiments, the synergist comprises a thiol substituent. In other embodiments, the synergist is an alkyl iodide. According to one or more embodiments, the synergist comprises thioglycolic acid, 2-mercaptoethanol, or a combination thereof. In embodiments, the synergist is present in the corrosion inhibitor formulation at a concentration of from 0.1 to 20 weight percent, 0.2 to 20 weight percent, 0.3 to 20 weight percent, 0.5 to 20 weight percent, 1 to 20 weight percent, 1 to 15 weight percent, 1 to 10 weight percent, 2 to 10 weight percent, 2 to 8 weight percent, or 2 to 6 weight percent.

In embodiments, the corrosion inhibitor formulation includes a secondary solvent. The secondary solvent may act to clean the metal for adsorption of an active component. In embodiments, the secondary solvent comprises ethylene glycol, ethylene diamine, or combinations thereof. According to one or more embodiments, the secondary solvent is present in the corrosion inhibitor formulation at a concentration of from 0.1 to 20 weight percent, 0.2 to 20 weight percent, 0.3 to 20 weight percent, 0.5 to 20 weight percent, 1 to 20 weight percent, 1 to 15 weight percent, 1 to 10 weight percent, 1 to 8 weight percent, 1 to 6 weight percent, or 2 to 5 weight percent.

According to one or more embodiments, the corrosion inhibitor formulation includes a surfactant. In embodiments, the surfactant comprises an ethoxylated alcohol. According to one or more embodiments, the ethoxylated alcohol comprises a carbon chain length of C₅ to C₂₀, C₈ to C₁₈, C₁₀ to C₁₅, or C₁₂ to C₁₄. According to one or more embodiments, the surfactant is present in the corrosion inhibitor formulation at a concentration of from 0.1 to 10 weight percent, 0.1 to 5 weight percent, 0.1 to 2 weight percent, 0.2 to 2 weight percent, 0.2 to 1.5 weight percent, 0.2 to 1 weight percent, 0.2 to 0.8 weight percent, 0.2 to 0.6 weight percent, 0.3 to 0.6 weight percent, or 0.4 to 0.6 weight percent.

In embodiments, the corrosion inhibitor formulation includes a coupling agent. The coupling agent may act to mitigate phase separation of components in the corrosion inhibitor formulation. The risk of phase separation may be especially acute in environments with a large temperature range. According to one or more embodiments, the coupling agent is an alkyl imino dipropionic acid sodium salt.

In some embodiments, the corrosion inhibitor formulation includes an ethoxylated amine. The ethoxylated amine may facilitate film formation and neutralize acids present in the environment. In embodiments, the ethoxylated amine is present in the corrosion inhibitor formulation at a concentration of from 0.1 to 10 weight percent, 0.1 to 5 weight percent, 0.2 to 5 weight percent, 0.3 to 5 weight percent, 0.3 to 3 weight percent, 0.3 to 2 weight percent, 0.5 to 2 weight percent, 0.5 to 1.5 weight percent, 0.7 to 1.5 weight percent, or 0.8 to 1.2 weight percent.

According to one or more embodiments, the corrosion inhibitor formulation includes a supporting component. The supporting component may act cooperatively, along with the active component, to mitigate corrosion. In some embodiments, the supporting component mitigates sweet corrosion in wet environments containing both CO₂ and H₂S. According to one or more embodiments, the supporting component comprises imidazoline. In embodiments, imidazoline is present in the corrosion inhibitor formulation at a concentration of from 0.1 to 20 weight percent, 0.2 to 20 weight percent, 0.3 to 20 weight percent, 0.4 to 20 weight percent, 0.5 to 20 weight percent, 1 to 20 weight percent, 1 to 15 weight percent, 1 to 10 weight percent, 2 to 10 weight percent, 2 to 8 weight percent, or 2 to 6 weight percent.

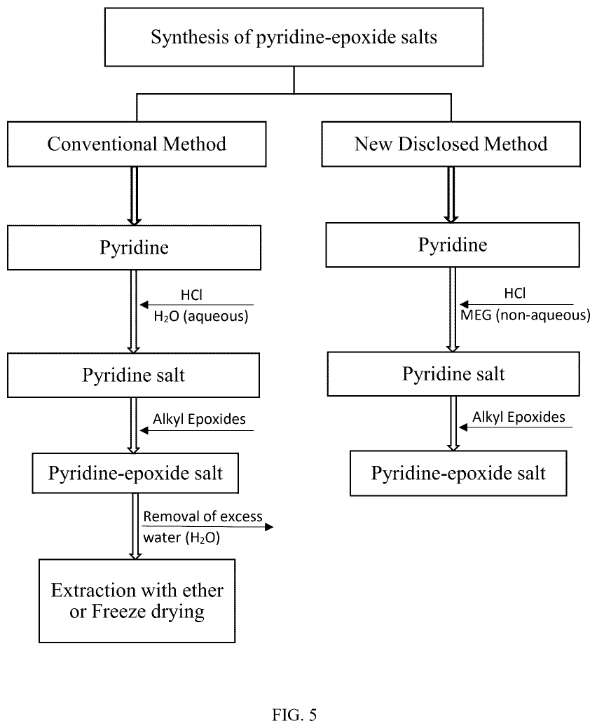

In embodiments, a process for inhibiting corrosion includes contacting a metallic surface with a corrosion inhibitor formulation according to embodiments described herein. According to embodiments, the metallic surface comprises steel. In some embodiments, the metallic surface comprises carbon steel. In embodiments, the metallic surface may be contacted with a solution containing the corrosion inhibitor formulation at a concentration by weight of 0.5 parts per million to 500 parts per million, 0.5 parts per million to 200 parts per million, 0.5 parts per million to 100 parts per million, 0.5 parts per million to 50 parts per million, 1 part per million to 50 parts per million, 1 part per million to 40 parts per million, 2 part per million to 40 parts per million, 2 part per million to 30 parts per million, 3 part per million to 30 parts per million, 3 part per million to 20 parts per million, 5 part per million to 20 parts per million, or 5 part per million to 15 parts per million. In embodiments, the metallic surface may be contacted with the corrosion inhibitor formulation at least one time, at least two times, at least three times, at least four times, or even at least five times. It should be understood that when the metallic surface is contacted with the corrosion inhibitor formulation multiple times, each contacting may be accomplished using the same or different concentrations of the compound described herein. Moreover as shown in FIG. 5, these formulations can be utilized for corrosion inhibition without first separating the pyridine-based salt compound out of the non-aqueous solvent.

Many oil and gas processing facilities such as gas oil separation plants and pipelines include metallic surfaces exposed to sour well fluids. Thus, adding the corrosion inhibitor formulation described herein to the sour well fluids may mitigate corrosion of the metallic surfaces. Thus, according to one or more embodiments, a process for inhibiting corrosion includes adding the corrosion inhibitor formulation described herein to a well fluid to effect a concentration by weight of the formulation in the well fluid of from 0.5 parts per million to 500 parts per million, 0.5 parts per million to 200 parts per million, 0.5 parts per million to 100 parts per million, 0.5 parts per million to 50 parts per million, 1 part per million to 50 parts per million, 1 part per million to 40 parts per million, 2 part per million to 40 parts per million, 2 part per million to 30 parts per million, 3 part per million to 30 parts per million, 3 part per million to 20 parts per million, 5 part per million to 20 parts per million, or 5 part per million to 15 parts per million.

Corrosion inhibitor formulations described herein may exhibit a higher corrosion inhibition efficiency than conventional corrosion inhibitor formulations. In embodiments, corrosion inhibitor formulations described herein may exhibit a corrosion inhibition efficiency that is at least 5%, at least 10%, at least 15%, at least 20%, at least 25%, or at least 30% greater than conventional corrosion inhibitor formulations. Because embodiments of the corrosion inhibitor formulations described herein exhibit a greater corrosion inhibition efficiency than conventional corrosion inhibitor formulations, a lower dosage of the corrosion inhibitor formulations described herein may provide a metallic surface with the same or even greater protection from corrosion than a higher dosage of a conventional corrosion inhibitor formulation. As such, corrosion inhibitor formulations described herein may be more efficient and cost-effective than conventional corrosion inhibitor formulations.

According to a first aspect, a formulation for inhibiting corrosion comprises a solution including a pyridine salt compound of Formula (I) dissolved in a non-aqueous solvent wherein R₁-R₆, independently comprise hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide; and X and Y independently comprise a heteroatom selected from oxygen, nitrogen, or sulfur, or a heterocarbyl comprising one or more heteroatoms selected from oxygen, nitrogen, or sulfur.

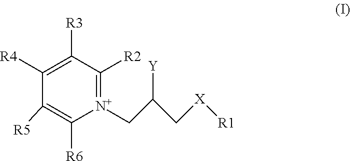

According to a second aspect, either alone or in combination with any other aspect, the formulation comprises a pyridine salt compound of Formula (1), wherein R₂, R₃, R₄, R₅, and R₆ are independently hydrogen; and R₁ is an unsubstituted C₅ to C₁₅ alkyl.

According to a third aspect, either alone or in combination with any other aspect, the formulation comprises the compound of Formula (I), wherein the compound is substantially soluble in water.

According to a fourth aspect, either alone or in combination with any other aspect, the compound has an average molecular mass ranging from 100 m/z to 1500 m/z.

According to a fifth aspect, either alone or in combination with any other aspect, the compound includes Formula (I) wherein the non-aqueous reaction solvent comprises at least one alcohol.

According to a sixth aspect, either alone or in combination with any other aspect, the compound includes Formula (I) wherein the alcohol comprises one or more of monethylene glycol (MEG), diethylene glycol monoethyl ether (DGME), or 2-butoxyethanol.

According to a seventh aspect, either alone or in combination with any other aspect, the formulation further comprises at least one of a synergist, a nonionic surfactant, imidazoline, a coupling agent, an ethoxylated amine, and water.

According to an eighth aspect, either alone or in combination with any other aspect, the pyridine salt compound is present at a concentration of from 0.5% to 60% weight percent; the water is present at a concentration of from 0% to 90% weight percent; the imidazoline is present at a concentration of from 0.1% to 20% weight percent; and the synergist comprises thioglycollic acid, 2-mercaptoethanol, or a mixture thereof.

According to a ninth aspect, either alone or in combination with any other aspect, a process for inhibiting corrosion comprises contacting a metallic surface with the formulation of claim 1.

According to a tenth aspect, either alone or in combination with any other aspect, wherein the metallic surface comprises steel.

According to an eleventh aspect, either alone or in combination with any other aspect, wherein the process is performed under wet sour crude conditions.

According to a twelfth aspect, either alone or in combination with any other aspect, a method of making a pyridinium salt-based solution comprises reacting a pyridine comprising Formula (II) or a salt thereof, with an epoxide comprising Formula (III) in the presence of an acid and a non-aqueous solvent to produce the pyridinium salt-based solution, comprising a pyridine salt compound of Formula (I) dissolved in the non-aqueous solvent, wherein R₂, R₃, R₄, R₅, and R₆ are independently hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide; R₁ is a substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, haloalkyl, azidoalkyl, amide, amino acid, or peptide; X and Y independently comprise a heteroatom selected from oxygen, nitrogen, or sulfur, or a heterocarbyl comprising one or more heteroatoms selected from oxygen, nitrogen, or sulfur.

According to a thirteenth aspect, either alone or in combination with the eleventh aspect, R₂, R₃, R₄, R₅, and R₆ are independently hydrogen; and R₁ is an unsubstituted C₅ to C₁₅ alkyl.

According to a fourteenth aspect, either alone or in combination with any other aspect, the non-aqueous reaction solvent comprises one or more alcohols.

According to a fifteenth aspect, either alone or in combination with any other aspect, the alcohol comprises at least one of monoethylene glycol, diethylene glycol monoethyl ether, or 2-butoxyethanol.

According to a sixteenth aspect, either alone or in combination any other aspect, the the acid is hydrochloric acid, sulfuric acid, phosphoric acid, hydrobromic acid, acetic acid, nitric acid, or a combination thereof.

According to a seventeenth aspect, either alone or in combination with any other aspect, the method further comprises preparing a formulation comprising the pyridinium salt-based solution without first separating the pyridine-based salt compound out of the non-aqueous solvent.

### EXAMPLES

Using embodiments described above, an exemplary corrosion inhibitor was prepared and used according to the following examples. The examples are illustrative in nature, and should not be understood to limit the subject matter of the present disclosure.

The following compositions were used in the Examples below.

All solvents including the pyridine, 1,2-epoxydodecane, monoethylene glycol, diethylene glycol monomethyl ether, 2-butoxyethanol, hydrochloric acid (37%), hydrobromic acid (37%), dichloromethane, dimethyl sulfoxide-D6, deuterated chloroform, deuterated water, and diethyl ether were purchased from Sigma-Aldrich.

### Example 1—Synthesis of Inventive Corrosion Inhibitor Examples

Corrosion Inhibitor 1 (CI-1), Corrosion Inhibitor 2 (CI-2), Corrosion Inhibitor 3 (CI-3), and Corrosion Inhibitor 4 (CI-4) were achieved substantially as depicted in Scheme I:

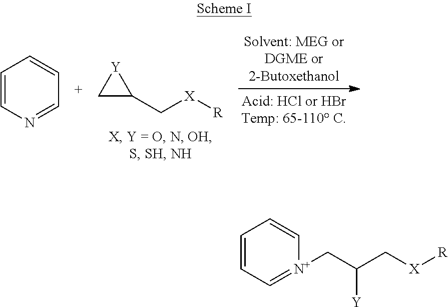

Briefly, pyridine (1.5 moles) and acid (1 mole, 37% solution in water) as listed in Table 1 below were added to a round bottom flask. The resulting mixture was purged with nitrogen and stirred at room temperature (25° C.) for 10 minutes. To the mixture, 1 mole of solvent as listed in Table 1 was added followed by an alkyl epoxide (1 mole) comprising either 1,2-epoxydodecane or dodecyl and tetradecyl glycidyl ether and the resulting mixture was stirred for 30 minutes and then heated at 100° C. for up to 6 hours before being cooled to room temperature for characterization. The reaction mixture turned brown with the conversion of the reaction mixture to corrosion inhibitor. The Corrosion Inhibitors CI-1, CI-2, CI-3, and CI-4 were prepared using the general reactants and solvents shown in Table 1.

Now, referring to comparative corrosion inhibitor 1 (CCI-1), CCI-1 was synthesized using the conventional synthesis approach for pyridinium-epoxide salts as shown in FIG. 5. Similar to CI-1, pyridine (1.5 mmol) and acid (1 mmol) were added to a round bottom flask, purged with nitrogen, and stirred at room temperature (25° C.) for 10 minutes. Then, 1,2-epoxydodecane (1 mmol) was added to the flask along with a volume of water equivalent to the volume of the added hydrochloric acid. The reaction mixture was stirred again for 30 minutes, then heated to 100° C. for 15 hours. At the end of the elapsed time, the excess pyridine was removed from the final solution via rotary evaporation. Diethyl ether was added to precipitate the final 1-(2-hydroxydodecyl)pyridinium compound as a white color material which was further dried at 50° C. in an oven to get a white powder.

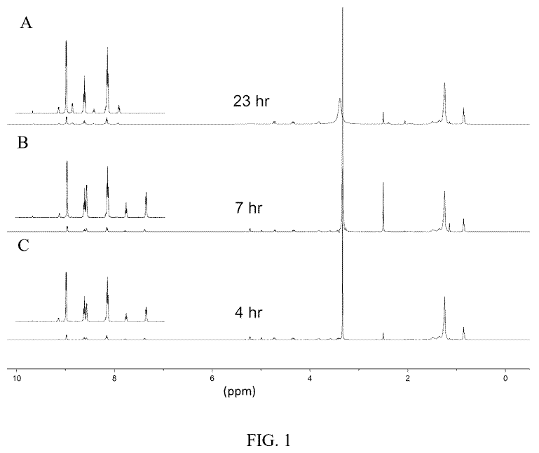

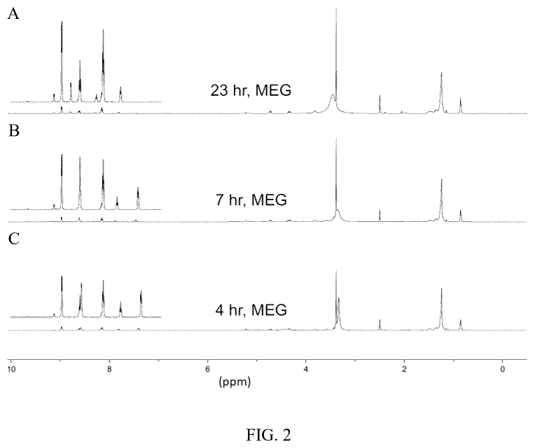

The synthesized corrosion inhibitors were characterized using proton nuclear magnetic resonance (¹H NMR) spectroscopy. Specifically, a sample of the pyridinium salt of epoxide in respective solvent was collected and dissolved in deuterated chloroform (CDCl₃) for NMR analyses and deuterated dimethyl sulfoxide (DMSO-d₆) and deuterated water (D₂O) were used as a reference for the chemical shift. A Varian 500 MHz VNMRS spectrometer and a JEOL 500 MHz NMR spectrometer were utilized to obtain spectra using appropriate acquisition parameters. The ¹H NMR analysis confirmed the structures of the pyridinium salt compounds comprising 1-(2-hydroxydodecyl) pyridinium bromide or 1-[3-(Dodecyloxy/tetradecyloxy)-2-hydroxypropyl]pyridinium chloride. FIG. 1 provides the ¹H NMR spectrum of the active component of the 1-(2-hydroxydodecyl) pyridinium salt prepared in water at 23 hours (FIG. 1A), 7 hours (FIG. 1B), and 4 hours (FIG. 1C). FIG. 2 provides the ¹H NMR spectrum of the corrosion inhibitor comprising 1-(2-hydroxydodecyl) pyridnium salt prepared in monoethylene glycol at 23 hours (FIG. 2A), 7 hours (FIG. 2B), and 4 hours (FIG. 2C).

A portion of each corrosion inhibitor was dissolved in methanol to a final concentration of 0.1 mg/mL and then characterized using Ion Trap Mass Spectrometry (IT-MS). An ion trap mass spectrometer equipped with an electrospray ion source (ESI) in positive mode was used. A syringe was used to infuse the samples at a flow rate 2 μL/min and the drying temperature was set to 200° C. The mass spectra were acquired in the 70-2200 amu mass range. The mass calibration was assessed with an electrospray tune mix diluted in 1:100 methanol.

TABLE 1
General Components used to Synthesize Corrosion Inhibitors

| Ex- | | | | |
|---|---|---|---|---|
| ample | Solvent | Alkyl Epoxide | Acid | Product |
| CI-1 | monoethylene | 1,2- | HBr | 1-(2-hydroxy dodecyl) |
| | glycol | Epoxydodecane | | pyridinium salt |
| | | | | 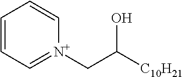 |
| CI-2 | diethylene glycol | 1,2- | HBr | 1-(2-hydroxy dodecyl) |
| | monomethyl ether | Epoxydodecane | | pyridinium salt |
| | | | | 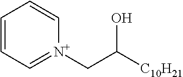 |
| CI-3 | diethylene glycol | 1,2- | HBr | 1-(2-hydroxy dodecyl) |
| | monomethyl ether | Epoxydodecane | | pyridinium salt |
| | | | | 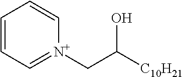 |
| CI-4 | monoethylene | Dodecyl and | HCl | 1-[3- |
| | glycol | tetradecyl glycidyl | | (Dodecyloxy/tetradecyloxy)-2- |
| | | ether | | hydroxypropyl] pyridinium salt |
| | | | | 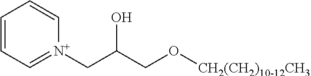 |

Referring to FIG. 1, the ¹H NMR spectrum of Active Component 1 (1-(2-hydroxy dodecyl)pyridinium salt) dissolved in water shows the pyridinium protons having a chemical shift of 9.01 ppm, 8.78 ppm, and 8.27 ppm. The protons on the carbon alpha to the pyridinium have a chemical shift of 5.00 ppm and 4.63 ppm. The proton on the carbon beta to the pyridinium and alpha to the hydroxy group has a chemical shift of 4.25 ppm. The protons on the carbon gamma to the pyridinium and beta to the hydroxy group have a chemical shift of 1.72 ppm. The remainder of the CH₂ protons are at 1.48 ppm while the CH₃ protons are present at 1.06 ppm.

Referring to FIG. 2, the ¹H NMR spectrum of Active Component 1 (1-(2-hydroxy dodecyl) pyridinium salt) dissolved in monoethylene glycol shows the pyridinium protons having a chemical shift of 9.01 ppm, 8.78 ppm, and 8.27 ppm. The protons on the carbon alpha to the pyridinium have a chemical shift of 5.00 ppm and 4.63 ppm. The proton on the carbon beta to the pyridinium and alpha to the hydroxy group has a chemical shift of 4.25 ppm. The protons on the carbon gamma to the pyridinium and beta to the hydroxy group have a chemical shift of 1.72 ppm. The remainder of the CH₂ protons are at 1.48 ppm while the CH₃ protons are present at 1.06 ppm.

From FIG. 1 and FIG. 2 it can be seen that despite the different reaction media, the compounds structures are identical, thus confirming the effectiveness of the disclosed synthesis method in maintaining the same original structure.

### Example 2—Synthesis of Corrosion Inhibitor Formulations

Corrosion Inhibitor Formulation 1 (CIF-1) and Corrosion Inhibitor Formulation 2 (CIF-2) were prepared using the general components shown in Table 2 and Table 3, respectively. Comparative Corrosion Inhibitor Formulation 1 (CIF-1) was prepared using the conventional synthesis approach for pyridinium-epoxide salts as shown in FIG. 5. The identity of the Active Compound in each Corrosion Inhibitor Formulation is shown in Table 4. The identity of the Active Compound in CCIF-1 is imidazoline. The identity of the Active Compound in CIF-1 and CIF-2 is Corrosion Inhibitor 4 (CI-4) which comprises 1-[3-(Dodecyloxy/tetradecyloxy)-2-hydroxypropyl]pyridinium-salt. The Inactive Compounds of CIF-1 include an ethoxylated amine and an alcohol. The Inactive Compounds of CIF-2 includes an alcohol. Corrosion tests under simulated and controlled dynamic field conditions were performed using a four-liter high temperature and high pressure (HTHP) autoclave rotating cage made from C-276 alloy, which withstands harsh corrosive environments.

TABLE 2
Composition of Corrosion Inhibitor Formulation 1 (CIF-1)

| Component | Weight (%) |
|---|---|
| Water | 77.0 |
| Thioglycollic Acid | 1.4 |
| 2-Merceptoethanol | 0.95 |
| Ethylene Glycol | 4.0 |
| 2-Butoxyethanol | 7.0 |
| Surfactant (Nonionic) | 0.3 |
| Ethoxylated Amine | 0.41 |
| Coupling Agent | 0.44 |
| Imidazoline | 1.0 |
| Corrosion Inhibitor 4 | 7.5 |

TABLE 3
Composition of Corrosion Inhibitor Formulation 2 (CIF-2)

| Component | Weight (%) |
|---|---|
| Ethylene Glycol | 37.5 |
| 2-Butoxyethanol | 7.5 |
| Corrosion Inhibitor 4 | 55.0 |

TABLE 4
Identity of the Active Component in
Each Corrosion Inhibitor Formulation

| Corrosion Inhibitor | Active Component |
|---|---|
| CIF-1 | Corrosion Inhibitor 4 |
| CIF-2 | Corrosion Inhibitor 4 |
| CCIF-1 | Imidazoline |

### Example 2—Performance of Corrosion Inhibitor Formulations

Briefly, carbon steel C-1018 coupons were cleaned and degreased following ASTM G1. The coupons were positioned in a fixed cage made from polyether ether ketone (PEEK), which was then mounted in the autoclave. The autoclave was purged with N₂ to remove dissolved oxygen and then the autoclave was pressurized to 250 psi with a gas comprising 7 mole % CO₂, 3 mole % H₂S, and 90 mole % N₂ and heated to a temperature of 83° C. (182° F.). To simulate field conditions, a 1:1 mixture of kerosene and water having a pH of 6.6 was prepared and mixed with 10 ppm of the corrosion inhibitor formulation to form a brine. The brine was stirred at room temperature and after 2 hours, was removed and the remaining water was used in the autoclave for the final test. The pressure was maintained using high purity nitrogen gas. The cage was rotated at 400 RPM for 24 hours. In all experiments, the corrosion inhibitor formulations were injected immediately after fixing the coupons in the autoclave. The geochemical analysis of the water is shown in Table 5. The concentration of the corrosion inhibitor formulation in the mixture for each respective test is also shown in Table 5.

TABLE 5
Water Geochemical Analysis

| Analyte | Concentration (mg/L) |
|---|---|
| Na | 29,500 |
| Ca | 8,210 |
| Mg | 1,080 |
| Cl | 61,800 |
| SO₄ | 1,300 |
| HCO₃ | 662 |
| Total Dissolved Solids | 102,552 |

As demonstrated in Table 6, CIF-1 and CIF-2 exhibit significantly superior corrosion inhibition efficiency relative to CCIF-1, which is a conventional corrosion inhibitor formulation used to inhibit corrosion in gas oil separation plants in a wet sour environment in the oil and gas industry, at 10 ppm dosage of corrosion inhibitor. Furthermore, CIF-1 and CIF-2 exhibit a decreased rate of corrosion relative to CCIF-1 at 10 ppm dosage of corrosion inhibitor. Although CIF-1 and CIF-2 demonstrate comparable inhibition efficiency and corrosion rate, it is noted that CIF-1 exhibited superior performance to CIF-2. Moreover, inventive examples CIF-1 and CIF-2 may be utilized for corrosion inhibition without separating the pyridine-based salt compound out of the non-aqueous solvent, so it is easier to produce while also yielding improved corrosion inhibition. Without being bound by theory, it is also believed that a corrosion inhibitor formulation comprising a pyridinium salt in non-aqueous media as described herein exhibits improved performance with respect to corrosion rates and corrosion inhibition. Without being bound by theory, it is also believed that a corrosion inhibitor formulation comprising the pyridinium salt compound of Formula (I), non-aqueous solvent, and at least one of a synergist, a nonionic surfactant, imidazoline, a coupling agent, an ethoxylated amine, and water may exhibit improved performance to formulations comprising the pyridinium salt compound of Formula (I) and non-aqueous solvent, with respect to corrosion rates and corrosion inhibition. Moreover, the method as described herein provides improved reaction kinetics with a shorter cycle time, thus, lowering the costs and commercialization of the final product.

The corrosion rate (CR) in mils per year was calculated for each corrosion inhibitor formulation using equation (1):

$$C R = \frac{Δ W \times 22 , 300}{D \times A \times T} \times 100 \% , \qquad ( 1 )$$

where ΔW is the weight loss of coupon in milligrams (mg), D is the density of the carbon steel coupons (7.89 g/cm³), A is the area of the exposed coupon (7.86 square inches) and T is the exposure time (24 hours). The corrosion rate of each corrosion inhibitor formulation is provided in Table 5.

The corrosion inhibition efficiency (IE %) of each corrosion inhibitor formulation was calculated using equation (2):

$$IE \% = \frac{( C {R}_{blank} - C {R}_{i n h i b i t o r} )}{C {R}_{blank}} \times 1 \; 00 \% , \qquad ( 2 )$$

where CR_{blank} is the corrosion rate without inhibitor and CR_{inhibitor} is the corrosion rate with the inhibitor. The corrosion inhibition efficiency of each corrosion inhibitor formulation is provided in Table 5

TABLE 6
Corrosion Inhibition Efficiency of
Corrosion Inhibitor Formulations

| Corrosion | | | Corrosion |
|---|---|---|---|
| Inhibitor | Concentration | Corrosion Rate | Inhibition |
| Formulation | (ppm) | (mils per year) | Efficiency (%) |
| CIF-1 | 10 | 4.5 | 72.5 |
| CIF-2 | 10 | 5.0 | 68.9 |
| CCIF-1 | 10 | 9.6 | 40.3 |

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” a second component, it is contemplated that, in embodiments, the first component “consists” or “consists essentially of” that second component.

It should be understood that any ranges provided herein include the endpoints unless stated otherwise.

It should be understood that any two quantitative values assigned to a property may constitute a range of that property, and all combinations of ranges formed from all stated quantitative values of a given property are contemplated in this disclosure.

It is also noted that recitations herein of “at least one” component, element, etc., should not be used to create an inference that the alternative use of the articles “a” or “an” should be limited to a single component, element, etc.

For the purposes of describing and defining the presently disclosed technology it is noted that the terms “substantially” and “about” are utilized herein to represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

What is claimed is:

1. A formulation for inhibiting metal corrosion comprising:
a solution comprises a pyridine salt compound of Formula (I) dissolved in a non-aqueous solvent 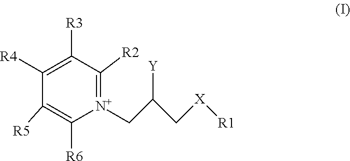
wherein R₁-R₆, independently comprise hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide; and
X and Y independently comprise a heteroatom selected from oxygen, nitrogen, or sulfur, or a heterocarbyl comprising one or more heteroatoms selected from oxygen, nitrogen, or sulfur.

2. The formulation of claim 1, wherein:
R₂, R₃, R₄, R₅, and R₆ are independently hydrogen; and
R₁ is an unsubstituted C₅ to C₁₅ alkyl.

3. The formulation of claim 1, wherein the compound is substantially soluble in water.

4. The formulation of claim 1, wherein the compound has an average molecular mass ranging from 100 m/z to 1500 m/z.

5. The formulation of claim 1, wherein the non-aqueous reaction solvent comprises at least one alcohol.

6. The formulation of claim 5, wherein the alcohol comprises one or more of monoethylene glycol (MEG), diethylene glycol monoethyl ether (DGME), 2-butoxyethanol.

7. The formulation of claim 1, further comprising at least one of a synergist, a nonionic surfactant, imidazoline, a coupling agent, an ethoxylated amine, and water.

8. The formulation of claim 7, wherein:
the pyridine salt compound is present at a concentration of from 0.5% to 60% weight percent;
the water is present at a concentration of from 0% to 90% weight percent;
the imidazoline is present at a concentration of from 0.1% to 20% weight percent; and
the synergist comprises thioglycollic acid, 2-mercaptoethanol, or a mixture thereof.

9. A process for inhibiting corrosion comprising contacting a metallic surface with the formulation of claim 1.

10. The process of claim 9, wherein the metallic surface comprises steel.

11. The process of claim 9, wherein the process is performed under wet sour crude conditions.

12. A method of making a pyridinium salt-based solution comprising:
reacting a pyridine comprising Formula (II): 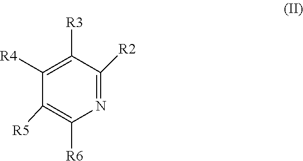
or a salt thereof, with an epoxide comprising Formula (III): 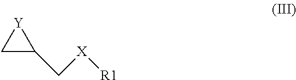
in the presence of an acid and a non-aqueous solvent to produce the pyridinium salt-based solution, comprising a pyridine salt compound of Formula (I) dissolved in the non-aqueous solvent, wherein
R₂, R₃, R₄, R₅, and R₆ are independently hydrogen, substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, aminoalkyl, haloalkyl, azidoalkyl, amide, amino acid, or peptide;
R₁ is a substituted or unsubstituted C₁ to C₂₄ alkyl, substituted or unsubstituted alkylaryl, substituted or unsubstituted aryl, substituted or unsubstituted heteroaryl, substituted or unsubstituted alkenyl, substituted or unsubstituted alkynyl, sulfonate, aryl sulfonate, C₁ to C₂₄ alkyl sulfonate, taurine, carboxylate, amine, alkylamine, arylamine, alkylammonium, arylammonium, sulfonamide, halogen, hydroxy, amide, nitro, cyano, azide, O-alkyl, S-alkyl, silyl, trialkylsilyl, O-silyl, haloalkyl, alkylsulfhydryl, trifluoromethyl, hydrazide, substituted or unsubstituted aryl, heteroaryl, or heterocyclic alkynyl, carboxyalkyl, haloalkyl, azidoalkyl, amide, amino acid, or peptide;
X and Y independently comprise a heteroatom selected from oxygen, nitrogen, or sulfur, or a heterocarbyl comprising one or more heteroatoms selected from oxygen, nitrogen, or sulfur.

13. The method of claim 11, wherein:
R₂, R₃, R₄, R₅, and R₆ are independently hydrogen; and
R₁ is an unsubstituted C₅ to C₁₅ alkyl.

14. The method of claim 11, wherein the non-aqueous reaction solvent comprises one or more alcohols.

15. The method of claim 13, wherein the alcohol comprises at least one of monoethylene glycol (MEG), diethylene glycol monoethyl ether (DGME), or 2-butoxyethanol.

16. The method of claim 11, wherein the acid is hydrochloric acid, sulfuric acid, phosphoric acid, hydrobromic acid, acetic acid, nitric acid, or a combination thereof.

17. The method of claim 11, further comprising preparing a formulation comprising the pyridinium salt-based solution without first separating the pyridine-based salt compound out of the non-aqueous solvent.
